(19) United States
(12) Patent Application Publication
(10) Pub. No.: US 2008/0131517 A1
(43) Pub. Date: Jun. 5, 2008

(54) Time-sustained-release formulations comprising a beta-blocker

(76) Inventors: Abdel Fawzy, Somerville, NJ (US); George Bobotas, Tarpon Springs, FL (US)

Correspondence Address: ARENT FOX LLP, 1050 CONNECTICUT AVENUE, N.W., SUITE 400, WASHINGTON, DC, 20036, US

(21) Appl. No.: 11/896,616

(22) Filed: Sep. 4, 2007

Related U.S. Application Data
(60) Provisional application No. 60/841,496, filed on Sep. 1, 2006.

(51) Int. Cl. A61K 9/14; A61K 31/138

(52) U.S. Cl. 424/494; 424/489; 424/490; 514/652

(57) ABSTRACT

The present invention relates to compositions and methods of treating human subjects with a beta-adrenergic receptor blocking agent (“beta-blocker”) provided in a time-sustained-release delivery system. The time-sustained-release drug delivery systems includes at least three populations of beads, where each population of beads includes a beta-blocker. The beads may be selected from immediate-release beads, enteric-release beads, sustained-release beads, and time-sustained-release beads. The beta-blocker may be selected from acebutolol, atenolol, betaxolol, bisoprolol, esmolol, metoprolol, nebivolol, butoxamine, carteolol, carvedilol, labetalol, nadolol, oxprenolol, penbutolol, propranolol, pindolol, sotalol, and timolol. According to presently preferred embodiments, the beta-blocker is propranolol. The dosage forms of the present invention are useful for treating conditions including hypertension, angina pectoris due to coronary atherosclerosis, hypertrophic subaortic stenosis, congestive heart failure, arrhythmias, angina, anxiety, glaucoma, migraines, esophageal varices, alcohol withdrawal syndrome, irregular heartbeat, tachycardia, tremor, and neuroleptic-induced akathisia. They are also useful in the prophylaxis of migraine headaches.

### BACKGROUND OF THE INVENTION

1. Field of the Invention

The present invention relates to compositions and methods for treating human subjects with a beta-adrenergic receptor blocking agent (“beta-blocker”) provided in a time-sustained-release delivery system.

2. Description of the Related Art

There are three types of beta receptors present in the human body. Hormones and neurotransmitters stimulate the sympathetic nervous system by acting on these receptors.

β1-receptors are located mainly in the heart, and when they are activated by adrenaline (epinephrine), or noradrenaline (norepinephrine), they cause an increased heart rate by acting on the cardiac pacemaker cells. β2-receptors are located throughout the body, but mainly in the lungs, muscles, and blood vessels of skeletal muscle. They cause vasodilation, thereby allowing more blood to flow to the muscles, and reducing total peripheral resistance. These beta receptors tend to work with adrenaline (epinephrine), but not noradrenaline (norepinephrine). β3-receptors are less well characterized, but are believed to be present in adipose tissue, and are thought to have a role in the regulation of lipid metabolism.

Beta-blockers or beta-adrenergic blocking agents are a class of drugs that block the action of epinephrine and/or norepinephrine on the beta-adrenergic receptors in the body. Activation of β1 receptors by epinephrine increases the heart rate and the blood pressure, and the heart consumes more oxygen. Drugs that block these β1 receptors have the reverse effect: they lower the heart rate and blood pressure. In addition, beta-blockers prevent the release of renin, a hormone produced by the kidneys that causes constriction of blood vessels. Drugs that block β2 receptors generally have a calming effect, and are used to treat conditions such as anxiety/panic disorders, migraines, portal hypertension/esophageal varices, and alcohol withdrawal syndrome. Many beta-blockers affect both β1 and β2 receptors; these are termed non-selective beta-blockers. Selective beta-blockers primarily affect β1-receptors, but they gradually become less selective at higher doses.

Beta-blockers are useful for treating a variety of cardiovascular conditions including high blood pressure (hypertension), congestive heart failure (CHF), abnormal heart rhythms (arrhythmias), and chest pain (angina). Beta-blockers are sometimes used in heart attack patients to prevent future heart attacks. Beta-blockers may also be used to treat other diseases including anxiety, glaucoma, migraine, esophageal varices, and alcohol withdrawal syndrome, among others. Beta-blockers are also used to correct irregular heartbeat, suppress the symptoms of hyperthyroidism (tachycardia, tremor), treat tremors and neuroleptic-induced akathisia (restlessness or need to keep moving caused by certain medicines used to treat nervousness or mental and emotional disorders), and treat pheochromocytoma (when administered together with an alpha-receptor blocking agent). Because they lower the heart rate, beta-blockers have been used by some marksmen to provide more aiming time between heart beats. Some performers use beta-blockers to avoid stage fright and tremor during auditions and performances. Beta-blockers have also been found to decrease nocturnal melatonin release.

Commonly-used β1-antagonists include Sectral (acebutolol), Tenormin (atenolol), Kerlone (betaxolol), Zebeta (bisoprolol), Brevibloc (esmolol), Lopressor/Toprol-XL (metoprolol), and nebivolol. An example of a β2-antagonist is butoxamine. Non-selective beta-blockers include Cartrol (carteolol), Coreg (carvedilol), Normodyne/Trandate (labetalol), Corgard (nadolol), Trasicor (oxprenolol—available only in Canada), Levatol (penbutolol), Inderal (propranolol), Inderal-LA (propranolol), Visken (pindolol), Betapace (sotalol), and Blocadren (timolol).

Propranolol [1-(isopropyl amino)-3-(1-naphthyloxy)-2-propanol] reduces the rate and force of contraction of the heart, and decreases the rate of conduction of impulses through the conducting system, thereby reducing the response of the heart to stress and exercise. These properties are used in the treatment of angina in an effort to reduce the oxygen consumption and increase the exercise tolerance of the heart. Propranolol is also used in the treatment of cardiac arrhythmias to block adrenergic stimulation of cardiac pacemaker potentials, in the long-term treatment of hypertension, and in the treatment of migraine and anxiety.

Propranolol is normally administered as propranolol hydrochloride tablets. One currently-available long-acting capsule formulation of propranolol is INDERAL® LA, which is manufactured by Wyeth Pharmaceuticals Inc. Another extended-release propranolol hydrochloride composition is InnoPran® XL, which is manufactured by Reliant Pharmaceuticals, Inc.

U.S. Pat. No. 4,138,475 discloses controlled release oral formulations comprising coated spheroids of propranolol or a pharmaceutically acceptable salt thereof. Each spheroid is coated with a mixture of 80 to 100% by weight of ethylcellulose, preferably having a viscosity of 50 cps at 20° C., 20 to 0% by weight of hydroxypropyl methylcellulose, and optionally up to 20% plasticizer based on the total weight of the membrane. Prior to membrane coating, these spheroids comprise 40 to 65% by weight propranolol or a pharmaceutically acceptable salt thereof, and 35 to 60% by weight of microcrystalline cellulose. The ratio of ethylcellulose to hydroxypropylcellulose and coating thickness depend upon the desired controlled release characteristics.

U.S. Pat. No. 4,957,745 discloses the art of making a controlled release formulation of a salt of metoprolol comprising a multitude of metoprolol cores prepared by layering the drug onto inert silicon dioxide beads, wherein the core is coated with a metoprolol permeable membrane of essentially ethylcellulose or a mixture of hydroxypropyl methylcellulose and ethylcellulose, the ratio of ethylcellulose to hydroxypropyl methylcellulose depending upon the desired control release characteristics.

Many therapeutic agents are most effective when made available at a constant rate at or near the absorption site. However, there are instances where maintaining a constant blood level of a drug is not desirable. A pulsatile or timed-release delivery system is capable of providing one or more pulses at predetermined time points after a controlled time lag, or at specific sites in the body.

The incidence of many cardiovascular diseases varies predictably in time over 24 hours, i.e., in a circadian rhythm fashion (See, e.g., Y. A. Anwar and W. B. White, Chronotherapeutics for Cardiovascular Disease, Drugs 1998, 55, pp 631-643, which is incorporated herein by reference). For example, an early morning rapid increase in both acute myocardial infarction and systolic blood pressure has been reported in well-controlled studies. In such cases, administration of a different kind of unit dosage form which delivers the drug in higher concentrations during the time of greatest need, typically during the early morning hours, and in lesser concentrations when the need is less, such as during late evening and early sleep hours.

There are only a few orally-applicable pulsatile release systems. Ishino et al. disclose a dry-coated tablet form in Chemical Pharm. Bull., Vol. 40 (11), 3036-041 (1992). U.S. Pat. No. 4,851,229, U.S. Pat. No. 5,011,692, U.S. Pat. No. 5,017,381, U.S. Pat. No. 5,229,135, and U.S. Pat. No. 5,840,329 all disclose preparation of pulsatile release systems. Related systems are disclosed in U.S. Pat. No. 4,871,549, and U.S. Pat. Nos. 5,260,068, 5,260,069, and 5,508,040.

U.S. Pat. No. 5,567,441 discloses a pulsatile release system consisting of pellets coated with delayed release or water insoluble polymeric membranes incorporating hydrophobic water insoluble agents or enteric polymers to alter membrane permeability. U.S. Pat. No. 5,837,284 discloses a dosage form which provides an immediate release dose of methylphenidate upon oral administration, followed by one or more additional doses spread over several hours.

U.S. Pat. No. 6,627,223 discloses multiparticulate dosage forms having timed, pulsatile release characteristics, wherein a series of pulses of drug release occur several hours after administration, with or without an immediate release pulse upon oral administration. The dosage forms have an active core, and the first coat surrounding the core is an enteric polymer, and the second coat is a combination of water insoluble and enteric polymers. The dosage forms may contain two or more drug particles with different release characteristics obtained by varying the coatings surrounding the active core. The dosage forms may be used to deliver cardiovascular agents.

U.S. Pat. No. 6,500,454 discloses timed, sustained-release drug delivery systems for propranolol. The systems include a unit dosage form including one or more timed, sustained-release bead populations, each comprising a propranolol core, where each bead population releases the therapeutic agent as a rapid or sustained release pulse after a predetermined delay. The beads have an active core, and the first coating surrounding the core is a sustained-release polymer, and the second or outer coating is a combination of water insoluble and enteric polymers. The systems release propranolol so that the resulting plasma concentration of the drug is highest during the early morning hours, to minimize the risk of strokes/heart attacks. The dosage forms may also include immediate release cores, in addition to the dual-coated cores.

U.S. Published Application No. 2004/0126427 A1 discloses extended release dosage forms for propranolol hydrochloride, including two populations of propranolol-containing particles, where one population provides a rapid-release profile (i.e., substantially complete release within 60 minutes), and the other population provides a sustained-release profile over a period of 24 hours. The unit dosage forms are provided as a capsule containing immediate-release beads and sustained-release beads of propranolol, where the sustained-release beads are obtained by providing a coating on the immediate-release beads, the coating containing a water-insoluble polymer or a mixture of a water-insoluble polymer and a water-soluble polymer. The extended release dosage forms are suitable for oral administration, and may be used in the treatment of cardiovascular disease.

None of these dosage forms described above provides a time-sustained-release formulation for beta-blockers/beta-adrenergic agents in accordance with the present invention.

There is clearly a great need in the art for extended-release/pulsatile-release release formulations for beta-blockers, and methods for making same. Methods of treating various conditions using the formulations are also needed.

### SUMMARY OF THE INVENTION

The present invention overcomes the above-mentioned problems, as well as others, by providing a delivery system and method to administer beta-blockers to subjects in a pulsatile or time-sustained-release fashion. One aspect of the present invention discloses a system and method to deliver a therapeutically-effective amount of sustained-release beta-blocker to a human subject to treat heart-related disorders, including hypertension, angina pectoris due to coronary atherosclerosis, hypertrophic subaortic stenosis, cardiac arrhythmias, and to prevent myocardial infarction. Another aspect of the present invention relates to a system and method to deliver a therapeutically-effective amount of sustained-release beta-blocker for use in the prophylaxis of migraine headaches. In some embodiments, the present invention is directed to delivering a therapeutically effective amount of time-sustained-release beta-blocker in an oral dosage form once daily.

In some embodiments, the present invention is directed to time-sustained-release drug delivery systems for beta-blockers, said systems including at least three populations of beads, each population of beads comprising a beta-blocker. The beads may be selected from immediate-release beads, enteric-coated beads, sustained-release beads, and time-sustained-release beads. The beta-blocker may be selected from acebutolol, atenolol, betaxolol, bisoprolol, esmolol, metoprolol, nebivolol, butoxamine, carteolol, carvedilol, labetalol, nadolol, oxprenolol, penbutolol, propranolol, pindolol, sotalol, and timolol. According to presently preferred embodiments, the beta-blocker is propranolol.

In other embodiments, the present invention is directed to method of preparing time-sustained-release drug delivery systems for beta-blockers, said methods including the steps of blending together at least three populations of beads, each population of beads comprising a beta-blocker. The beads may be selected from immediate-release beads, enteric-coated beads, sustained-release beads, and time-sustained-release beads. The blended beads are then filled in a pharmaceutically-acceptable delivery system. The beta-blocker may be selected from acebutolol, atenolol, betaxolol, bisoprolol, esmolol, metoprolol, nebivolol, butoxamine, carteolol, carvedilol, labetalol, nadolol, oxprenolol, penbutolol, propranolol, pindolol, sotalol, and timolol. According to presently preferred embodiments, the beta-blocker is propranolol.

In still other embodiments, the present invention relates to methods of administering beta-blockers to a patient, including administering an oral dosage form that includes at least three populations of beads, each population of beads comprising a beta-blocker, and where the oral dosage form provides time-sustained-release of the beta-blocker to the patient, thereby providing therapeutic relief. The beads may be selected from immediate-release beads, enteric-coated beads, sustained-release beads, and time-sustained-release beads. The beta-blocker may be selected from acebutolol, atenolol, betaxolol, bisoprolol, esmolol, metoprolol, nebivolol, butoxamine, carteolol, carvedilol, labetalol, nadolol, oxprenolol, penbutolol, propranolol, pindolol, sotalol, and timolol. According to presently preferred embodiments, the beta-blocker is propranolol. Preferably, the therapeutic relief is provided during a period of time when it is most needed by a patient. According to further preferred embodiments, the methods of administration are useful for relieving conditions including hypertension, angina pectoris due to coronary atherosclerosis, hypertrophic subaortic stenosis, congestive heart failure, arrhythmias, angina, anxiety, glaucoma, migraines, esophageal varices, alcohol withdrawal syndrome, irregular heartbeat, tachycardia, tremor, and neuroleptic-induced akathisia. They are also useful in the prophylaxis of migraine headaches.

Additional advantages and novel features of the invention will also become more apparent to those skilled in the art upon examination of the following or upon learning by practice of the invention.

### BRIEF DESCRIPTION OF THE DRAWINGS

FIG. 1 is a semi-log plot comparing the plasma concentration of propranolol after administration of an oral dosage form in accordance with the present invention versus INDERAL® LA. The dosage forms were administered to healthy volunteers following a high-fat meal.

FIG. 2 is a linear plot comparing the plasma concentration of propranolol after administration of an oral dosage form in accordance with the present invention versus INDERAL® LA. The dosage forms were administered to healthy volunteers following a high-fat meal.

FIG. 3 is a semi-log plot comparing the plasma concentration of propranolol after administration of an oral dosage form in accordance with the present invention versus INDERAL® LA. The dosage forms were administered to fasting healthy volunteers.

FIG. 4 is a linear plot comparing the plasma concentration of propranolol after administration of an oral dosage form in accordance with the present invention versus INDERAL® LA. The dosage forms were administered to fasting healthy volunteers.

Other features of the present invention will become apparent from the following detailed description considered in connection with the accompanying drawings, which disclose multiple embodiments of the present invention. It should be understood, however, that the figures are designed for the purpose of illustration only and not as a definition of the limits of the invention. Additional advantages and novel features of the invention will also become apparent to those skilled in the art upon examination of the following or upon learning by practice of the invention.

### DETAILED DESCRIPTION OF THE PREFERRED EMBODIMENTS

The present invention relates to time-sustained-release drug delivery systems comprising at least three beads, each containing at least one beta-blocker, methods of making same, and their use in treating conditions that include, but are not limited to, hypertension, angina pectoris due to coronary atherosclerosis, hypertrophic subaortic stenosis, congestive heart failure, arrhythmias, angina, anxiety, glaucoma, migraines, esophageal varices, alcohol withdrawal syndrome, irregular heartbeat, tachycardia, tremor, and neuroleptic-induced akathisia. They are also useful in the prophylaxis of migraine headaches. The at least one beta-blocker may be selected from acebutolol, atenolol, betaxolol, bisoprolol, esmolol, metoprolol, nebivolol, butoxamine, carteolol, carvedilol, labetalol, nadolol, oxprenolol, penbutolol, propranolol, pindolol, sotalol, and timolol. According to one presently preferred embodiment, the drug delivery systems and methods of the present invention include the non-specific beta-blocker propranolol. Additional compounds useful in treating these and related conditions may also be beneficially coadministered with the inventive drug delivery systems and related pharmaceutical formulations, or may be provided in a unit dose form therewith. For example, when used in the treatment of hypertension, the drug delivery systems and methods of the present invention may also beneficially be administered in conjunction with a thiazide diuretic, such as hydrochlorothiazide. According to another presently preferred embodiment, hydrochlorothiazide may be incorporated into the drug delivery systems of the present invention in an amount that is therapeutically effective in the treatment of hypertension, preferably about 25 mg, although doses up to 100 mg may be used.

### 1. Time-Sustained-Release Beta-Blocker Delivery Systems

Dosage forms incorporating the coated drug-containing beads in accordance with the invention may take a variety of forms. In one embodiment, the formulation may include at least three different types of beta-blocker beads, each having different release characteristics, i.e., combinations of modified-release beads with distinctly different lag times and release rates, with or without an immediate release bead, to form said time-sustained-release drug delivery system. Multicoated beads containing two or more drugs can also be combined to obtain synergistic efficacy and improve patient compliance. As used herein, the term “beads” is considered to be synonymous with cores, particles, spheroids, etc., as well as other terms used to describe particulate dosage forms, regardless of whether the active ingredients are provided as a coating, or are contained evenly throughout.

The present invention may incorporate now known or future known beta-blockers in an amount generally recognized as safe. Generally, the effect of the beta-blocker is dose dependent, i.e., the higher the dose, the greater the therapeutic affect. However, the effect of each beta-blocker is different, and therefore the level of therapeutic effect of beta-blocker cannot be necessarily be directly correlated to the level of therapeutic effects of other beta-blockers. However, those of ordinary skill in the art would understand the correct dosage to be given to a particular subject, based on experience and the seriousness of the condition.

The dosage forms of the present invention may incorporate any amount of beta-blocker that is effective in the treatment of the various conditions for which the dosage forms are useful in accordance with the present invention. For example, when the beta-blocker is propranolol, the dosage forms preferably incorporate a dose of from 60 to 160 mg of propranolol, with particularly preferred dosage forms incorporating doses of 60 mg, 80 mg, 120 mg, or 160 mg of propranolol. The time-sustained-release dosage forms of the present invention preferably are administered once-a-day. When the beta-blocker is propranolol, starting doses of 80 mg daily are preferred, and the daily dose may be increased over time to achieve therapeutic effectiveness in the treatment of the particular condition from which the patient is suffering. For example, when used in accordance with methods of treating hypertension, the therapeutic dose of propranolol is preferably from 120 mg to 160 mg daily, although the daily dose necessary to treat hypertension will vary for each patient, and higher doses (e.g., 640 mg) may be required to achieve full hypertensive response. When used in accordance with methods of treating angina pectoris, the therapeutic dose of propranolol is preferably about 160 mg daily, although the daily dose necessary to treat angina pectoris may be as high as 320 mg daily.

### A. Immediate-Release Beads Containing Beta-Adrenergic Blocking Agents

The beads containing the beta-blocker that are used in the novel dosage forms of the present invention may be comprised of an inert particle, such as a commercially-available non-pareil sugar sphere, which is then coated with a beta-blocker, or a granule that has been extruded and spheronized such that it contains the beta-blocker evenly dispersed throughout. The amount of beta-blocker provided in or on the beads will depend on the specific drug being used, and the dose that is desired. Generally, the beads will preferably contain about 35 to 75% by weight of the drug based on the total weight of the beads, most preferably 50 to 60% by weight. Those skilled in the art will be able to select an appropriate amount of beta-blocker for coating or incorporation into the beads to achieve the desired dosage form.

The beta-blocker may be selected from acebutolol, atenolol, betaxolol, bisoprolol, esmolol, metoprolol, nebivolol, butoxamine, carteolol, carvedilol, labetalol, nadolol, oxprenolol, penbutolol, propranolol, pindolol, sotalol, and timolol. According to presently preferred embodiments, the beta-blocker is propranolol.

Optional excipients may be used in the preparation of the immediate-release beta-blocker beads of the present invention. Excipients, for example, serve to solubilize, suspend, thicken, dilute, emulsify, stabilize, preserve, protect, color, and fashion the active ingredients into an applicable and efficacious preparation that is safe, convenient, and otherwise acceptable for use. When provided, the excipients are provided in an amount such that they do not deleteriously affect the delivery of the beta-blocker.

According to one embodiment, a solvent medium may be used to coat inert core particles with beta-blockers to form the immediate-release beads. The type of inert binder that is used to bind the beta-blockers to the inert particle is not critical, but usually water soluble or alcohol soluble binders are used. Binders such as polyvinylpyrrolidone (PVP), carboxyalkylcelluloses, polyethylene oxide, polysaccharides such as dextran, corn starch, hydroxypropyl methylcellulose (HPMC), hydroxypropylcellulose, may be used by dispersing them in water at a concentration of from about 0.5 to 5 weight %. According to a presently preferred embodiment, Povidone® (PVP) is used as a binder. Beta-blockers may be present in this coating formulation in solution form, or they may be suspended.

In another embodiment, the immediate-release beads may comprise matrices containing beta-blockers throughout, and may be prepared by granulation or by extrusion and spheronization. The beta-blockers, binders, and optionally other pharmaceutically-acceptable excipients may be blended together in a high shear granulator, such as Fielder granulator, or a fluid bed granulator, such as Glatt GPCG granulator, and granulated to form agglomerates by adding/spraying a granulating fluid such as water or alcohol, followed by drying. The wet mass can be extruded and spheronized to produce spherical particles (beads) using an extruder/marumerizer. In the matrix embodiment, the drug load can be as high as 90% by weight based on the total weight of the extruded or granulated beads. The preferred beta-blocker concentration varies from about 10 to 85 weight %, preferably from 35 to 75 weight %, and most preferably from 50 to 60 weight % of the beads.

In accordance with the present invention, the beads provide immediate release of the beta-blocker, comparable to other immediate-release beta-blocker compositions.

### B. Coatings to Provide Desired Release Characteristics

According to preferred embodiments of the present invention, the immediate-release beads containing beta-blockers are coated to provide the desired release characteristics. Particularly preferred coatings provide enteric release of the beta-blockers, sustained release of the beta-blockers, and/or time-sustained release of the beta-blockers. However, additional release-modifying coatings are envisioned in accordance with the present invention. The presently preferred coatings that may be applied to the immediate-release beads to provide desired release characteristics are described in greater detail below.

1. Enteric-Release Coated Beads

A preferred enteric-release coating composition according to the present invention includes polymers that are insoluble in the acidic environment of the stomach, but are soluble in the more neutral environment of the small intestine.

Representative examples of enteric polymers useful in the invention include esters of cellulose and its derivatives (e.g., cellulose acetate phthalate, hydroxypropyl methylcellulose phthalate, hypromellose phthalate, and hydroxypropyl methylcellulose acetate succinate), vinyl and its derivatives (e.g., polyvinyl acetate phthalate), and acrylic and its derivatives (e.g., pH-sensitive methacrylic acid-methamethacrylate copolymers), as well as natural materials (e.g., shellac). These polymers may be used singularly or in combination, either as a dry powder or an aqueous dispersion. A particularly preferred enteric-release polymer in accordance with the present invention is hypromellose phthalate.

The enteric coating of the present invention is preferably provided in an amount of from 4 to 16 weight %, more preferably from 6 to 14 weight %, and most preferably from 8 to 12 weight %, based on the total weight of the coated beads. However, those skilled in the art will appreciate that the amount of the enteric coating to be applied will vary based on the specific enteric polymer.

The outer coating comprising an enteric polymer may be applied to the beads using methods and techniques known in the art. Typically a suspension, emulsion, or solution of the polymeric coating is prepared as would be known in the art. In one embodiment of the present invention, water and acetone are used to solubilize the coating for application, and the solubilizing agents are removed during the coating process. The amount of fluidized polymeric coating required in the coating process may be readily calculated depending upon the amount of enteric polymer desired. The fluid sustained release polymer may be applied to the beads by a number of coating techniques known in the art. Examples of suitable coating devices include fluid bed coaters, pan coaters, etc.

The enteric polymer coating may be plasticized to provide improved physical and mechanical properties to the enteric coating, and to improve processing conditions during manufacturing. The choice of plasticizer is not critical, and it is within the capability of those skilled in the art to select plasticizers that are soluble and/or miscible with the enteric polymer, as well as to select quantities of plasticizer suitable for providing the desired handling characteristics. Examples of such plasticizers include acetyl triethyl citrate, dibutyl phthalate, tributyl citrate, triethyl citrate, acetyl tributyl citrate, propylene glycol, triacetin, polyethylene glycol and diethyl phthalate. A particularly preferred plasticizer in accordance with the present invention is diethyl phthalate. The plasticizer is preferably provided in an amount ranging from 0 to 5 weight %, preferably from 1 to 4 weight %, based on the total weight of the coated beads.

2. Sustained-Release Coated Beads

A preferred sustained-release coating composition according to the present invention includes polymers that are minimally soluble, and degrade at a predictable rate over time in the environment of the stomach and small intestine.

Representative examples of sustained-release polymers useful in the invention include cellulose derivatives (e.g., ethylcellulose, carboxy methylcellulose (CMC), hydroxypropyl methylcellulose (HPMC), polyvinylpyrrolidone, cellulose acetate, cellulose propionate, cellulose acetate butyrate, etc.), polymerized acrylates or copolymers of acrylic acid and methacrylic acid or esters of either monomer (e.g., poly(methyl methacrylate), poly(ethyl methacrylate), poly(butyl methacrylate), poly(isobutyl methacrylate), poly(isobutyl methacrylate), poly(phenyl methacrylate), etc.). The sustained-release coating may also be prepared using organosiloxanes (e.g., polydimethylsiloxane, polydiethylsiloxane, etc.). Use of other sustained-release coatings is also envisioned in accordance with the present invention (e.g., polyethylene, polypropylene, polyethylene oxide, polyvinyl acetate, polyvinyl chloride, etc.). Combinations of any of the above-recited sustained release coatings may be utilized. The desired sustained-release profile may be controlled by adjusting the sustained-release coating composition and thickness. A preferred sustained-release polymer in accordance with the present invention is ethylcellulose.

The sustained-release coating of the present invention is preferably provided in an amount of from 0.5 to 5 weight %, more preferably from 1 to 3 weight %, based on the total weight of the coated beads. However, those skilled in the art will appreciate that the amount of the sustained-release coating to be applied will vary based on the specific sustained-release polymer.

The outer coating comprising a sustained-release polymer may be applied to the beads using methods and techniques known in the art. Typically a suspension, emulsion, or solution of the polymeric coating is prepared as would be known in the art. In one embodiment of the present invention, water and acetone are used to solubilize the coating for application, and the solubilizing agents are removed during the coating process. The amount of fluidized polymeric coating required in the coating process may be readily calculated depending upon the amount of sustained release polymer desired. The fluid sustained release polymer may be applied to the beads by a number of coating techniques known in the art. Examples of suitable coating devices include fluid bed coaters, pan coaters, etc.

The sustained-release polymer coating may be plasticized to provide improved physical and mechanical properties to the enteric coating, and to improve processing conditions during manufacturing. The choice of plasticizer is not critical, and it is within the capability of those skilled in the art to select plasticizers that are soluble and/or miscible with the sustained-release polymer, as well as to select quantities of plasticizer suitable for providing the desired handling characteristics. Examples of such plasticizers include acetyl triethyl citrate, dibutyl phthalate, tributyl citrate, triethyl citrate, acetyl tributyl citrate, propylene glycol, triacetin, polyethylene glycol and diethyl phthalate. A particularly preferred plasticizer in accordance with the present invention is diethyl phthalate, which may be provided in an amount ranging from 0 to 5 weight %, preferably from 0.1 to 4 weight %, based on the total weight of the coated beads.

3. Time-Sustained-Release Coated Beads

A preferred time-sustained-release coating composition according to the present invention includes both enteric polymers and polymers that are minimally soluble, which result in a sustained release of beta-blocker that is delivered several hours after administration.

Time-sustained-release coatings are designed to release the beta-blocker slowly over a period of 4-16 hours after a 2-8 hour lag time. The time-sustained-release coatings are preferably produced by applying to immediate-release beads an inner layer of sustained-release polymer, and an outer layer of an enteric-release polymer or a blend of an enteric polymer and sustained-release polymer. Time-sustained-release coated beads may alternatively be produced by simultaneously applying a blend of enteric and sustained-release polymers.

The enteric and sustained-release polymers that are used to form the time-sustained-release coatings are any of those described above with respect to the enteric coatings and the sustained-release coatings. According to a preferred embodiment, the time-sustained-release coatings include hypromellose phthalate as the enteric polymer, and ethylcellulose as the sustained-release polymer. The enteric-release polymer is provided in an amount from 4 to 16 weight %, more preferably from 6 to 14 weight %, and most preferably from 8 to 12 weight %, based on the total weight of the coated beads. The sustained-release polymer is preferably provided in an amount from 4 to 16 weight %, preferably from 5 to 14 weight %, and most preferably about 6 to 12 weight %, based on the total weight of the coated beads. This amount can be as a single coating, or divided for multiple coatings.

The layer(s) comprising the time-sustained-release coating may be applied to the core using methods and techniques known in the art. Typically a suspension, emulsion, or solution of the polymeric coating is prepared as would be known in the art. In one embodiment of the present invention, water and acetone are used to solubilize the coating for application, and the solubilizing agents are removed during the coating process. The amount of fluidized polymeric coating required in the coating process may be readily calculated depending upon the amount of sustained release polymer desired. The fluid sustained-release polymer may be applied to the active core by a number of coating techniques known in the art. Examples of suitable coating devices include fluid bed coaters, pan coaters, etc.

The layer(s) comprising the time-sustained-release coating may include plasticizers to provide improved physical and mechanical properties to the coating layers, and to improve processing conditions during manufacturing. The choice of plasticizer is not critical, and it is within the capability of those skilled in the art to select plasticizers that are soluble and/or miscible with the enteric and sustained-release polymers, as well as to select quantities of plasticizer suitable for providing the desired handling characteristics. Examples of such plasticizers include acetyl triethyl citrate, dibutyl phthalate, tributyl citrate, triethyl citrate, acetyl tributyl citrate, propylene glycol, triacetin, polyethylene glycol and diethyl phthalate. A particularly preferred plasticizer in accordance with the present invention is diethyl phthalate, which may be provided in an amount ranging from 0 to 5 weight %, preferably from 1 to 4 weight %, based on the total weight of the coated beads.

### 2. Time-Sustained-Release Beta-Blockers Dosage Forms

The time-sustained-release beta-blocker compositions of the present invention may be prepared in any oral dosage form acceptable for use with the various types of beads of the present invention, although gelatin capsules are particularly preferred.

The oral dosage forms of the present invention are preferably gelatin capsules containing at least three types of beads comprising beta-blockers. The three types of beads are selected from immediate-release beads, enteric-release beads, sustained-release beads, and time-sustained-release beads. Particularly preferred are oral dosage forms including all four of these types of beads.

The dosage forms of the present invention preferably include immediate-release beads in an amount of from 0 to 25 weight %, more preferably from 3 to 20 weight %, and most preferably from 5 to 15 weight %. Enteric-coated beads are preferably provided in an amount of from 2.5 to 30 weight %, more preferably from 5 to 25 weight %, and most preferably from 10 to 20 weight %. Sustained-release beads are preferably provided in an amount of from 10 to 40 weight %, more preferably from 12.5 to 30 weight %, and most preferably from 15 to 25 weight %. Time-sustained-release beads are preferably provided in an amount of from 30 to 80 weight %, more preferably from 40 to 70 weight %, and most preferably from 45 to 65 weight %. All weight percentages are based on the total weight of beads provided, not including any weight attributable to a capsule or other means for containing the beads.

### 3. Methods of Treatment Using Time-Sustained-Release Beta-Blockers

The formulations of the present invention are particularly useful in controlling hypertension in patients suffering therefrom. Presently preferred methods of treatment involve administering the formulations of the present invention in an amount that is sufficient to provide an effective dose of a beta-blocker to the patient at bedtime. The time-sustained-release formulations of the present invention provide a time-sustained-release delivery of the beta-blocker such that the peak plasma concentration in the patient may be achieved in the early morning, when many patients are at a higher risk of heart attacks.

These methods of treatment are also useful for treating angina pectoris due to coronary atherosclerosis, and hypertrophic subaortic stenosis. They are also useful in the prophylaxis of migraine headaches.

It is also envisioned that the administration of these formulation may be included in treatment regimens designed to relieve/prevent other conditions, such as congestive heart failure, arrhythmias, angina, migraines, glaucoma, esophageal varices, alcohol withdrawal syndrome, esophageal varices, alcohol withdrawal syndrome, irregular heartbeat, tachycardia, tremor, neuroleptic-induced akathisia, and anxiety. Additional methods of treatment are also contemplated, and are within the scope of the present invention.

### EXAMPLES

Particularly preferred embodiments of the present invention will now be described with respect to the following non-limiting examples.

1. In Vitro Analysis of Rate of Propranolol Release

The release characteristics of the sustained-release beta-blocker formulations in accordance with the present invention may be measured by conducting in vitro dissolution testing. All in vitro testing herein was conducted according to Test 1 of the 2006 US Pharmacopeia 29 Official Monograph for Propranolol Hydrochloride Extended-Release Capsules, hereby incorporated by reference. This test utilizes Apparatus 1, 100 rpm, using 900 ml pH 1.2 buffer solution for 1.5 hours, then 900 ml pH 6.8 buffer solution for the remainder.

1.1 In Vitro Dissolution Characteristics of Formulation 1

The release characteristics of a first four-bead formulation are set forth in Table 1 below:

TABLE 1

| | % Propranolol |
|---|---|
| Time (hours) | (Label Claim) Released |
| 1.5 | 17% |
| 4 | 53% |
| 8 | 77% |
| 14 | 92% |
| 24 | 101% |

1.2 In Vitro Dissolution Characteristics of Formulation 2

The release characteristics of a second four-bead formulation are set forth in Table 2 below:

TABLE 2

| | % Propranolol |
|---|---|
| Time (hours) | (Label Claim) Released |
| 1.5 | 15% |
| 4 | 52% |
| 8 | 76% |
| 14 | 92% |
| 24 | 101% |

1.3 In Vitro Dissolution Characteristics of Formulation 3

The release characteristics of a third four-bead formulation are set forth in Table 3 below:

TABLE 3

| | % Propranolol |
|---|---|
| Time (hours) | (Label Claim) Released |
| 1.5 | 13% |
| 4 | 47% |
| 8 | 71% |
| 14 | 87% |
| 24 | 96% |

1.4 In Vitro Dissolution Characteristics of Formulation 4

The release characteristics of a fourth four-bead formulation are set forth in Table 4 below:

TABLE 4

| | % Propranolol |
|---|---|
| Time (hours) | (Label Claim) Released |
| 1.5 | 12% |
| 4 | 47% |
| 8 | 70% |
| 14 | 85% |
| 24 | 93% |

1.5 In Vitro Dissolution Characteristics of Formulation 5

The release characteristics of a fifth four-bead formulation are set forth in Table 5 below:

TABLE 5

| | % Propranolol |
|---|---|
| Time (hours) | (Label Claim) Released |
| 1.5 | 14% |
| 4 | 50% |
| 8 | 72% |
| 14 | 88% |
| 24 | 97% |

2. Comparison of Plasma Levels of Propranolol

The total plasma level of propranolol in the plasma of human subjects after administration of the dosage forms of the present invention was compared to the total level of propranolol in the plasma of human subjects after administration of a prior art formulation, Wyeth Pharmaceuticals' Inderal® LA 160 mg propranolol HCl sustained-release capsules.

2.1 6006 RelPran vs. Inderal LA

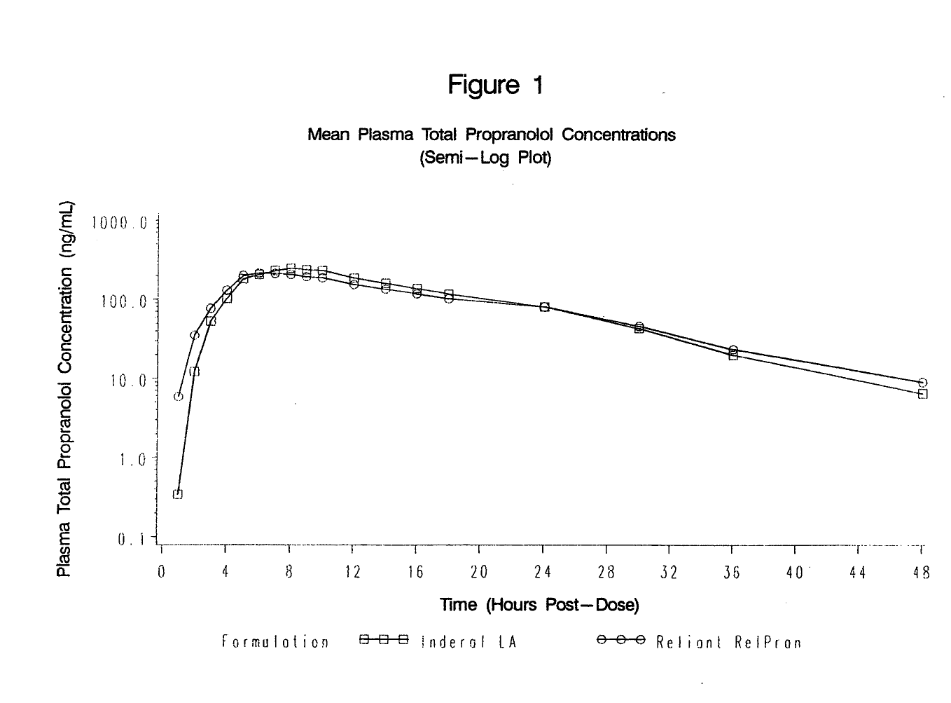

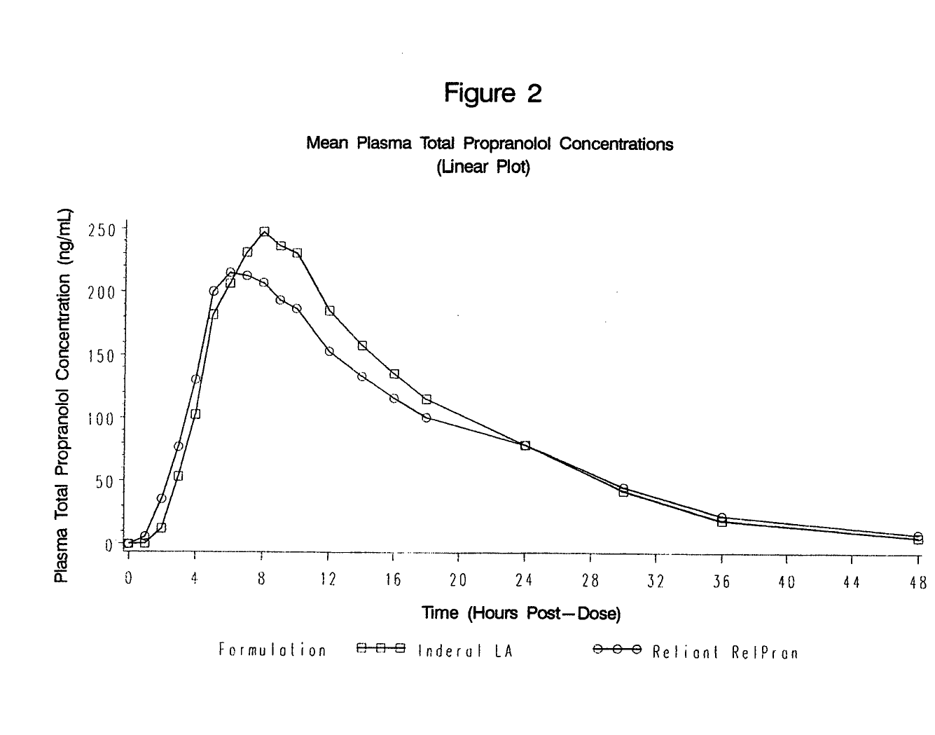

A comparative, randomized, single-dose, two-way crossover bioavailability study was conducted to compare the third four-bead formulation (designated 6006 RelPran) with Wyeth Pharmaceuticals' Inderal® LA 160 mg propranolol HCl sustained-release capsules in 72 healthy adult volunteers under fed (high-fat) conditions. Each subject received a single 160 mg dose of 6006 RelPran or Inderal® LA during each study period. A total of 67 subjects completed the study. Blood was drawn from the subjects, and the total propranolol concentrations in plasma were determined by Liquid Chromatography/Mass Spectrometry/Mass Spectrometry (LC/MS/MS). Plots comparing the mean plasma propranolol concentrations over a 48-hour period are provided in FIGS. 1 and 2. Additional data supporting the bioequivalence of 6006 RelPran and Inderal® LA are presented in Tables 6 and 7.

TABLE 6
Total Plasma Levels of Propranolol (6006 RelPran)

| | Ln AUC₀₋ₜ | Ln AUC_{0-∞} | Ln Cₘₐₓ |
|---|---|---|---|
| | (ng · h/mL) | (ng · h/mL) | (ng/mL) |
| Ratio of Least- | 95.1% | 96.7% | 94.0% |
| Square Means | | | |
| (A/B) % | | | |
| 90% CI | 91.4% | 93.4% | 87.8% |
| Lower Limit | | | |
| 90% CI | 98.9% | 100.1% | 100.5% |
| Upper Limit | | | |

TABLE 7
Total Plasma Levels of Propranolol

| Study | | | | Cₘₐₓ* | tₘₐₓ | | kel |
|---|---|---|---|---|---|---|---|
| Lot# | n | AUC₀₋ₜ* | AUC_{0-∞}* | (ng/mL) | (hrs) | T_{1/2} (hrs) | (1/hr) |
| 6006 | 67 | 3644.7 | 3726.5 | 245.1 | 8.2 | 6.4 | 0.121 |
| A94415 | | | | | | | |

*Geometric means

5.2 6008 RelPran vs. Inderal LA

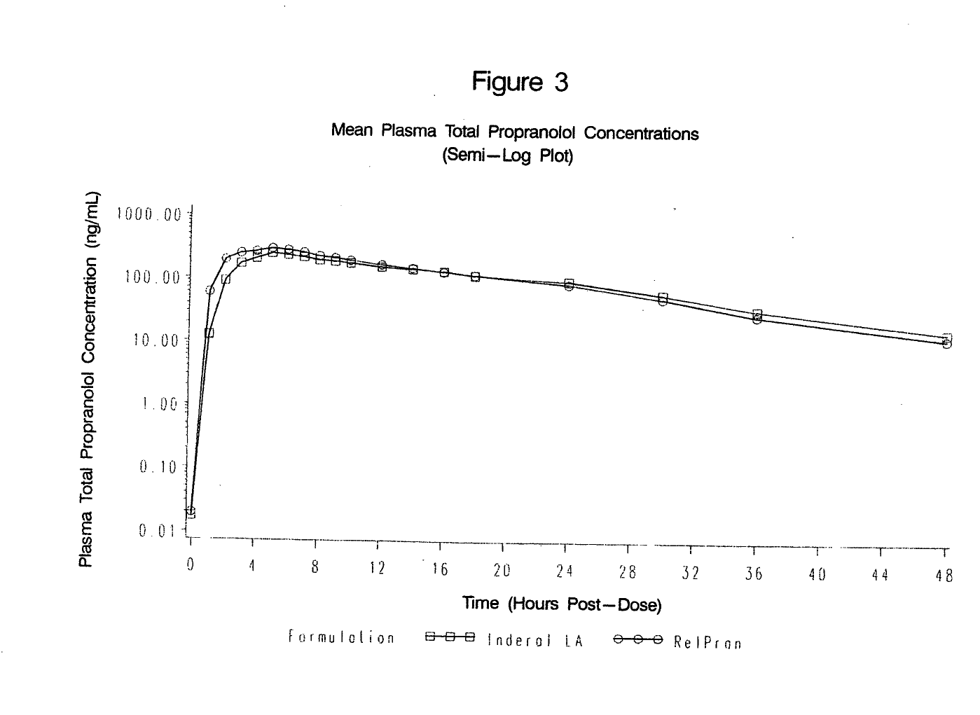

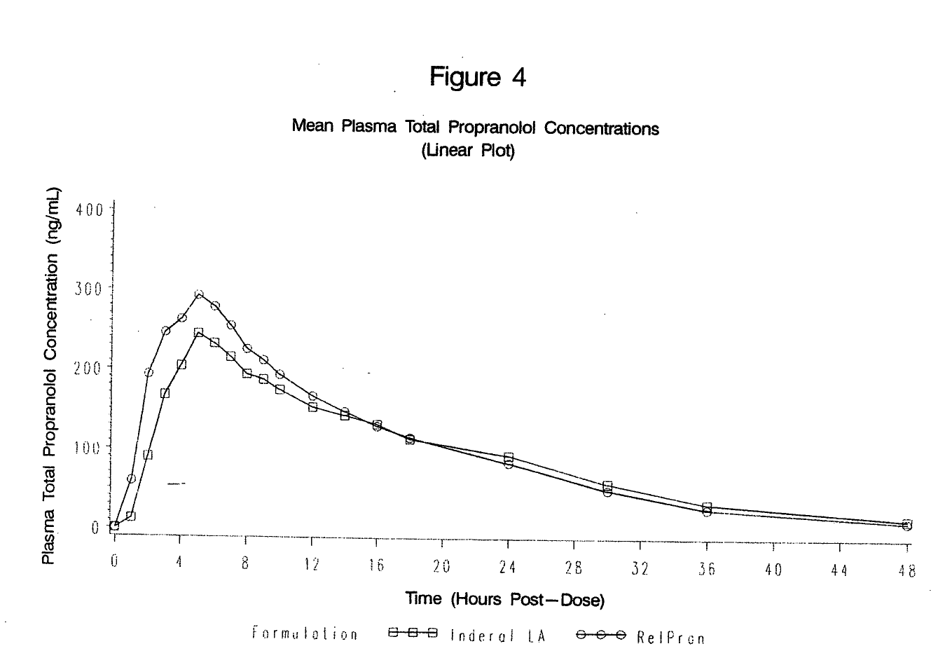

A comparative, randomized, single-dose, two-way crossover bioavailability study was conducted to compare the third four-bead formulation (designated 6008 RelPran) with Wyeth Pharmaceuticals' Inderal® LA 160 mg propranolol HCl sustained-release capsules in 150 healthy adult volunteers under fasting conditions. Each subject received a single 160 mg dose of 6008 RelPran or Inderal® LA during each study period. A total of 142 subjects completed the study. Blood was drawn from the subjects, and the total propranolol concentrations in plasma were determined by Liquid Chromatography/Mass Spectrometry/Mass Spectrometry (LC/MS/MS). Plots comparing the mean plasma propranolol concentrations over a 48-hour period are provided in FIGS. 3 and 4. Additional data supporting the bioequivalence of 6008 RelPran and Inderal® LA Data are presented in Tables 8 and 9.

TABLE 8
Total Plasma Levels of Propranolol (6008 RelPran)

| | Ln AUC₀₋ₜ | Ln AUC_{0-∞} | Ln Cₘₐₓ |
|---|---|---|---|
| | (ng · h/mL) | (ng · h/mL) | (ng/mL) |
| Ratio of Least- | 108.0 | 106.0 | 120.5 |
| Square Means | | | |
| (A/B) % | | | |
| 90% CI | 105.5 | 103.5 | 117.6 |
| Lower Limit | | | |
| 90% CI | 110.6 | 108.6 | 123.5 |
| Upper Limit | | | |

TABLE 9
Total Plasma Levels of Propranolol

| Study | | | | Cₘₐₓ* | tₘₐₓ | T_{1/2} | kel |
|---|---|---|---|---|---|---|---|
| Lot# | n | AUC₀₋ₜ* | AUC_{0-∞}* | (ng/mL) | (hrs) | (hrs) | (1/hr) |
| A94415 | 142 | 4085 | 4280 | 239 | 5.7 | 9.1 | 0.08590 |
| 6008 | 142 | 4411 | 4563 | 288 | 4.8 | 8.4 | .0919 |
| (PF370 | | | | | | | |
| EA0001) | | | | | | | |

*Geometric means presented

It will, of course, be appreciated that the above description has been given by way of example only and that modifications in detail may be made within the scope of the present invention.

Throughout this application, various patents and publications have been cited. The disclosures of these patents and publications in their entireties are hereby incorporated by reference into this application, in order to more fully describe the state of the art to which this invention pertains.

The invention is capable of considerable modification, alteration, and equivalents in form and function, as will occur to those ordinarily skilled in the pertinent arts having the benefit of this disclosure.

While the present invention has been described for what are presently considered the preferred embodiments, the invention is not so limited. To the contrary, the invention is intended to cover various modifications and equivalent arrangements included within the spirit and scope of the detailed description provided above.

What is claimed is:

1. A time-sustained-release drug delivery system for beta-blockers, said system comprising at least three populations of beads, wherein each population of beads comprises a beta-blocker.

2. The time-sustained-release drug delivery system of claim 1, wherein the populations of beads are selected from the group consisting of immediate-release beads, enteric-coated beads, sustained-release beads, and time-sustained-release beads.

3. The time-sustained-release drug delivery system of claim 1, wherein the beta-blocker is selected from the group consisting of acebutolol, atenolol, betaxolol, bisoprolol, esmolol, metoprolol, nebivolol, butoxamine, carteolol, carvedilol, labetalol, nadolol, oxprenolol, penbutolol, propranolol, pindolol, sotalol, and timolol.

4. The time-sustained-release drug delivery system of claim 1, wherein the beta-blocker is propranolol and the dose of propranolol is from 60 to 160 mg.

5. The time-sustained-release drug delivery system of claim 1, wherein the beads comprise about 10 to about 85% by weight of the beta-blocker, based on the total weight of the beads.

6. The time-sustained-release drug delivery system of claim 2, wherein the enteric-coated beads comprise an enteric coating comprising polymers selected from the group consisting of esters of cellulose and its derivatives, vinyl and its derivatives, acrylic and its derivatives, and natural materials.

7. The time-sustained-release drug delivery system of claim 6, wherein the enteric coating is provided in an amount from 4 to 16 weight % based on the total weight of the beads.

8. The time-sustained-release drug delivery system of claim 2, wherein the sustained-release beads comprise a sustained-release coating comprising polymers selected from the group consisting of cellulose derivatives, polymerized acrylates, copolymers of acrylic acid and methacrylic acid, esters of acrylic acid and methacrylic acid, organosiloxanes, polyethylene, polypropylene, polyethylene oxide, polyvinyl acetate, and polyvinyl chloride.

9. The time-sustained-release drug delivery system of claim 8, wherein the sustained-release coating is provided in an amount from 0.5 to 5 weight % based on the total weight of the beads.

10. The time-sustained-release drug delivery system of claim 2, wherein the time-sustained-release coating comprises enteric polymers selected from the group consisting of esters of cellulose and its derivatives, vinyl and its derivatives, acrylic and its derivatives, and natural materials, and sustained-release polymers selected from the group consisting of cellulose derivatives, polymerized acrylates, copolymers of acrylic acid and methacrylic acid, esters of acrylic acid and methacrylic acid, organosiloxanes, polyethylene, polypropylene, polyethylene oxide, polyvinyl acetate, and polyvinyl chloride.

11. The time-sustained-release drug delivery system of claim 10, wherein the enteric polymers are provided in an amount from 4 to 16 weight % based on the total weight of the beads, and the sustained-release polymers are provided in an amount from 4 to 16 weight % based on the total weight of the beads.

12. The time-sustained-release drug delivery system of claim 2, wherein the immediate-release beads are provided in an amount of from 0 to 25 weight %, the enteric-coated beads are provided in an amount of from 2.5 to 30 weight %, the sustained-release beads are provided in an amount of from 10 to 40 weight %, and the time-sustained-release beads are provided in an amount of from 30 to 80 weight %, based on the total weight of beads.

13. A method of preparing time-sustained-release drug delivery systems for beta-blockers, said method comprising the steps of blending together at least three populations of beads, wherein each population of beads comprises a beta-blocker.

14. The method of preparing time-sustained-release drug delivery systems of claim 13, wherein the populations of beads are selected from the group consisting of immediate-release beads, enteric-coated beads, sustained-release beads, and time-sustained-release beads.

15. The method of preparing time-sustained-release drug delivery systems of claim 13, further comprising the step of filling the blended beads in a pharmaceutically-acceptable delivery system.

16. The method of preparing time-sustained-release drug delivery systems of claim 13, wherein the beta-blocker is selected from the group consisting of acebutolol, atenolol, betaxolol, bisoprolol, esmolol, metoprolol, nebivolol, butoxamine, carteolol, carvedilol, labetalol, nadolol, oxprenolol, penbutolol, propranolol, pindolol, sotalol, and timolol.

17. A method of administering beta-blockers to a patient comprising the step of administering an oral dosage form that includes at least three populations of beads, wherein each population of beads comprises a beta-blocker, and the oral dosage form provides time-sustained-release of the beta-blocker to the patient, thereby providing therapeutic or prophylactic relief.

18. The method of administering beta-blockers to a patient of claim 17, wherein the populations of beads are selected from the group consisting of immediate-release beads, enteric-coated beads, sustained-release beads, and time-sustained-release beads.

19. The method of administering beta-blockers to a patient of claim 17, wherein the beta-blocker is selected from the group consisting of acebutolol, atenolol, betaxolol, bisoprolol, esmolol, metoprolol, nebivolol, butoxamine, carteolol, carvedilol, labetalol, nadolol, oxprenolol, penbutolol, propranolol, pindolol, sotalol, and timolol.

20. The method of administering beta-blockers to a patient of claim 17, wherein the method provides therapeutic relief of at least one condition selected from the group consisting of hypertension, angina pectoris due to coronary atherosclerosis, hypertrophic subaortic stenosis, congestive heart failure, arrhythmias, angina, anxiety, glaucoma, migraines, esophageal varices, alcohol withdrawal syndrome, irregular heartbeat, tachycardia, tremor, and neuroleptic-induced akathisia, or provides prophylaxis of migraine headaches.
